(19) United States
(12) Patent Application Publication
(10) Pub. No.: US 2024/0130830 A1
(43) Pub. Date: Apr. 25, 2024

(54) PORTABLE SYSTEM FOR ISOLATION AND REGULATION OF SURGICAL SITE ENVIRONMENTS

(71) Applicant: SURGIBOX INC., Cambridge, MA (US)

(72) Inventors: Debbie Lin Teodorescu, Cambridge, MA (US); Mike Horia Mihail Teodorescu, Cambridge, MA (US); Macauley Reardon Dupree Kenney, Cambridge, MA (US); Nathan Samuel Phipps, Watertown, MA (US)

(73) Assignee: SURGIBOX INC., Cambridge, MA (US)

(21) Appl. No.: 18/278,171

(22) PCT Filed: Nov. 8, 2021
(86) PCT No.: PCT/US2021/058496

§ 371 Date: Aug. 21, 2023

Related U.S. Application Data
(60) Provisional application No. 63/154,761, filed on Feb. 28, 2021.
(60) Provisional application No. 63/247,545, filed on Sep. 23, 2021.

(51) Int. Cl. A61B 90/40; A61G 10/00; A61G 10/02

(52) U.S. Cl. CPC A61B 90/40; A61G 10/005; A61G 10/02; A61B 2090/401

(57) ABSTRACT

Portable surgical systems for regulating intra-operative environments over surgical sites are disclosed. The surgical systems include a flexible surgical enclosure configured to be attached to the surgical site of a patient. The enclosure enables operators to perform surgery on the surgical site from inside the enclosure. The enclosure may further include a patient-limb-port configured to enable the patient to insert an arm or a leg into the enclosure so that a limb-surgical-site is disposed inside the enclosure. The surgical system may further include an active environment control unit with one or multiple sensors. The surgical system prevents outer environment contaminants from reaching the surgical site while providing a barrier protecting operators from exposure to blood generated during the surgery. The portable surgical system is used to perform surgery in environments other than operating rooms, such as outdoors, in tents, in cottages, and non-sterile rooms.

### CROSS REFERENCE TO RELATED APPLICATIONS

This application claims priority from and the benefit of the United States Provisional Patent Application No. 63/154,761 filed on Feb. 28, 2021 and titled “UTILITARIAN TASK-BASED CONTAINER AND INFLATABLE ISOLATION CHAMBER”, and the United States Provisional Patent Application No. 63/247,545 filed on Sep. 23, 2021 and titled “PORTABLE SYSTEM 10 FOR ISOLATION AND REGULATION OF SURGICAL SITE ENVIRONMENTS” which are hereby incorporated by reference for all purposes as if fully set forth herein.

### BACKGROUND OF THE INVENTION

### I. Field of The Invention

Exemplary embodiments of the present invention relate to a portable surgical system for regulating intra-operative environments over surgical sites and to methods for implementing and using the same.

### II. Discussion of the Background

Over 25% of the global disease burden requires surgical therapy, which could prevent over 18 million deaths per year. These range from obstetric complications to traumas to infections to cancer and beyond. Yet 2 billion people have no meaningful access to safe surgical care, and 2-3 billion more have access only to unsterile surgeries in contaminated environments, leading to disproportionate rates of surgical infections. Innovations in this field typically focus upon making operating rooms and operating room ventilation systems more mobile, such as in tent format. However, such systems remain costly to purchase and to maintain. Moreover, such systems are difficult to transport rapidly to remote areas. At the same time, over 85,000 medical providers are infected by patient bodily fluids annually, with 90% of infected providers worldwide having been exposed while working in low-resource settings. While personal protective equipment mitigates these risks to some extent, there is a definite trade-off between the level of protection and both the cost as well as the user comfort, which is well-documented to correspond to user compliance.

Exemplary embodiments of the present invention aim to address both challenges of patient and provider intraoperative exposure to infectious risks and airborne particulates by implementing an ultraportable, self-contained, passive and active, bilateral barrier against exchange of contaminants between incisions and the greater surgical area.

The above information disclosed in this Background section is only for enhancement of understanding of the background of the invention and therefore it may contain information that does not form any part of the prior art.

### SUMMARY OF THE INVENTION

Exemplary embodiments of the present invention provide a portable surgical system for regulating intra-operative environments over surgical sites. The portable surgical systems disclosed herein address both challenges of patient and operator intraoperative exposure to infectious risks. Additionally, the portable surgical systems herein protects both patient and operators from exposure to fluids (e.g., blood, and other bodily fluids) and airborne particulates (e.g., dust in the environment, spores, viruses, bacteria) incident to the surgical procedures.

The surgical system ensures that the surgical site is kept sterile by preventing contaminants from the outer environment (i.e., outside of the surgical enclosure) from reaching the surgical site. Also, the surgical system is configured to ensure that contaminants on other areas of the patient body are not reaching the surgical site. The surgical system provides a barrier protecting operators from exposure to contaminants (e.g., blood) generated during the surgery inside the enclosure. The portable surgical system may be used to perform surgery in environments other than operating rooms, such as in the field, outdoors, tents, cottages, residential rooms, etc.

The portable surgical system may include a flexible surgical enclosure configured to be attached to the body of a patient. The enclosure may include an incise-drape configured to be disposed on the torso of the patient so as to cover a torso-surgical-site of the patient if surgery is needed on the torso-surgical-site. The enclosure may further include a patient-limb-port configured to enable the patient to insert an arm or a leg into the enclosure so that a limb-surgical-site is disposed inside the enclosure if surgery is needed on patient's arm or leg.

The enclosure may further include one or more arm-ports and arm sleeves enabling an operator to access and to perform surgery on the torso-surgical-site or on the limb-surgical-site disposed inside the enclosure. The enclosure may further include one or more transparent layers enabling the operator to view the torso-surgical-site or the limb-surgical-site during the surgery. The surgical enclosure may include an adhesive-surface disposed around the incise-drape and attached to the patient around the surgical-site so as to create a seal. Upon removal of the incise-drape the surgical-site of the patient becomes included in the inside of the enclosure and accessible by the operator from the inside of the enclosure whereas other surface areas of the patient are disposed outside the enclosure. The surgical enclosure may be sterilized by various known methods in the art, such as gamma sterilization, gas sterilization, UV sterilization, etc. The packaging of the surgical enclosure may be designed according to a wide variety of methods such as to preserve sterility of the enclosure. The incise drape may be designed by a variety of methods known to the art such as to preserve an airtight environment comprising of the inside of the enclosure and the now attached to the enclosure patient surgical site, such as adhesives, belts, Velcro attachments, etc.

The surgical enclosure may include a fluids-reservoir configured to collect the unwanted blood and fluids generated in the enclosure during surgery. The fluids-reservoir is disposed on the lower part of the enclosure and may be made as a fold of the enclosure material. The fluids reservoir may comprise fill sensors as well as rulers or other visual measuring aids to indicate to the operator the amount of fluid lost during surgery. This may indicate blood loss during the procedure. The fluids-reservoir may also be used to improve visibility during the use of the surgical enclosure as unwanted fluids accumulate in the reservoir as opposed to remaining around the surgical site. The fluids-reservoir may be disposed in such a manner as fluid flow to be guided by gravity into the reservoir or actively managed such as through the use of a suction device that would dispose unwanted fluids into the reservoir in low-gravity environments. A suction line may be attached to the fluid reservoir through a controlled one-way valve system.

The portable surgical system may include an environmental control system configured to supply and control air flow and pressure inside the enclosure such as to ensure a sterile environment inside the enclosure and over the surgical sites. The environmental control system may include a fan, an air-filter, a pressure sensor configured to measure the pressure inside the enclosure, a control-system, and an air-tube disposed at least partially inside the enclosure. The air-tube is configured to receive air from the air-supply-system. The air-tube may include one or more outlets disposed inside the enclosure and configured to generate air-flow over the surgical site. The control-system may be configured to receive a series of pressure readings from the pressure sensor and to control the air pressure and air-flow in the enclosure to desired values. The control system may include one or several microprocessors with programs customized to maintain desired pressure, airflow, temperature, or other environmental parameters in the enclosure through sensor control loops. The control system may include one or several pressure control loops, one or several temperature control loops, one or several humidity control loops, and one or several air-flow control loops. In the latter case, the control system may maintain a different pressure in an inflatable frame that supports the environment control system than the pressure inside the surgical enclosure. The control system may adjust based on the environment parameters outside of the enclosure, such as through differential pressure, temperature, and airflow sensors that would maintain desired parameters inside the surgical enclosure environment irrespective of the outside temperature, pressure, and wind speed. This control system may mitigate outside environment conditions such as use at high altitude, use in low temperature conditions, or windy conditions, to name a few scenarios.

The surgical system may include a frame attached to the flexible surgical enclosure. The frame is configured to provide stability to the flexible surgical enclosure without obscuring the visibility through the surgical enclosure. The frame is configured to provide a tension over an axial length of the enclosure and to create inside the enclosure an operating volume enabling operators to perform surgery on the surgical sites. The frame may have a loop shape including two rigid spacer-segments interspaced by two flexible tensioner-segments. The tensioner-segments are configured to bend so that the frame assumes essentially a saddle shape. While deployed for operation, the flexible enclosure attached to the frame acts on the frame so as to keep the frame into the saddle shape which includes bent tensioner-segments. The enclosure may be attached to the frame via a plurality of attachment-means of adjustable length. The width and other dimensions of the enclosure may be adjusted by adjusting the length of the attachment-means. The frame may include a plurality of segments where at least some of the segments of the frame are configured to have adjustable lengths so that an operator can adjust the dimensions of the frame by adjusting the lengths of the segments.

The portable surgical system may further include one or more lights configured to illuminate the surgical-site and one or more cameras configured to image the surgical-site. The one or more lights may be LED strip lights disposed on the enclosure or incorporated into the enclosure.

The surgical system is configured to be used for performing surgery outdoors (e.g., wounded soldiers in the field, inhabitants of remote regions, rescue operations in wilderness, etc.) and in environments which lack the sterility of hospital operating room (e.g., tents, cottages, residential rooms, non-operating rooms in hospitals, etc.). The surgical system is configured to be portable, light, ergonomic and easy to install. The surgical system may be configured to be packed into a portable bag (e.g., backpack) so as to be easy to carry in the field.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 shows a perspective view of a portable surgical system disposed over the body of a patient subject to surgery which may be performed in an environment different than hospital facilities.

FIG. 2 shows a photograph of a prototype for an exemplary embodiment of the portable surgical system in FIG. 1.

FIG. 3(a) shows a view of an exemplary embodiment of the portable surgical system while used by an operator to perform surgery on a patient.

FIG. 3(b) shows another view of an exemplary embodiment of the portable surgical system while used by an operator to perform surgery on a patient.

FIG. 4 shows an oblique front-side perspective view of the frame and the surgical enclosure of an exemplary embodiment of a portable surgical system.

FIG. 5 shows a front-side view of the frame and surgical enclosure of an exemplary embodiment of a portable surgical system.

FIG. 6 shows a side view of the frame and surgical enclosure of an exemplary embodiment of a portable surgical system.

FIG. 7 shows an oblique back-side view of the frame and surgical enclosure of an exemplary embodiment of a portable surgical system.

FIG. 8 shows a back-side view of the frame and surgical enclosure of an exemplary embodiment of a portable surgical system.

FIG. 9 shows a top side view of the frame and surgical enclosure of an exemplary embodiment of a portable surgical system.

FIG. 10 shows a top view of the frame and surgical enclosure of an exemplary embodiment of a portable surgical system.

FIG. 11(a) shows a configuration of an attachment-means between the surgical enclosure and the frame in a disconnected state.

FIG. 11(b) shows a configuration of an attachment-means connecting/attaching the surgical enclosure and the frame.

FIG. 12(a) shows an exemplary embodiment of a frame prior to being attached to the surgical enclosure and prior to being tensioned.

FIG. 12(b) shows an exemplary embodiment of a frame in a tensioned state assumed while attached to the surgical enclosure.

FIG. 13 shows a surgical enclosure attached on a frame, the forces exerted upon the frame by the enclosure and the tension generated into the enclosure by the frame.

FIG. 14 shows the back side of the surgical enclosure and a frame while the frame is stretching the enclosure to a desired width via the attachment-means.

FIG. 15 shows an exemplary embodiment of a frame configured to have an adjustable length and width.

FIG. 16 shows an exemplary embodiment of a plurality of portable, packable frame modules configured to be easily assembled into a frame.

FIG. 17 shows an exemplary embodiment of a surgical system employing an inflatable-structure instead of a rigid frame.

FIG. 18 shows an exemplary embodiment of a surgical system employing an inflatable-structure disposed inside the enclosure instead of a rigid frame.

FIG. 19 shows an exemplary embodiment of a surgical system employing an inflatable-structure comprising rib-air-beams and top-air-beams.

FIG. 20 shows another exemplary embodiment of a surgical system employing an inflatable-structure comprising rib-air-beams and base-air-beams.

FIG. 21 shows an exemplary embodiment of a sleeve configured to be used by the operator to access the surgical site.

FIG. 22 shows another exemplary embodiment of a sleeve configured to be used by the operator to access the surgical site.

FIG. 23 shows an exemplary embodiment of a surgical system while used to operate on a hand or arm of a patient.

FIG. 24 shows an exemplary embodiment of a surgical system while used to operate on a leg or foot of a patient.

FIG. 25 shows an exemplary embodiment of a surgical system including an alternative technical design for the arm/leg port.

FIG. 26 shows an exemplary embodiment of a surgical system including an alternative technical design for the arm/leg port.

FIG. 27 shows an exemplary embodiment of a surgical system including an alternative technical design for the arm/leg port.

FIG. 28 shows an exemplary embodiment of a surgical system including an alternative technical design for the arm/leg port.

FIG. 29 shows an exemplary embodiment of a surgical system including a material port configured to enable instruments, trays, devices, and materials to be moved into and out of the surgical enclosure.

FIG. 30(a) shows a front side of a surgical system including an assembly of line ports.

FIG. 30(b) shows an exemplary embodiment of a line port assembly as in FIG. 30(a).

FIG. 30(c) shows a first layer of the assembly of line ports in FIG. 30(b).

FIG. 30(d) shows a second layer of the assembly of line ports in FIG. 30(b).

FIG. 31(a). shows an exemplary embodiment of a fluids-reservoir configured to collect unwanted fluids, such as blood, generated inside the enclosure during the surgery.

FIG. 31(b). shows a section/portion of the fluids-reservoir in FIG. 31(a).

FIG. 31(c). shows a section/portion of the fluids-reservoir in FIG. 31(a) including a scale.

FIG. 31(d). shows a section/portion of the fluids-reservoir in FIG. 31(a) including a strain-sensor.

FIG. 32 shows an exemplary embodiment of a bottom side of the surgical system including incise-drapes and adhesive regions.

FIG. 33 shows a surgical system including an environmental control system configured to generate air-flow inside the enclosure so as to create a sterile surgical environment.

### DETAILED DESCRIPTION

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity. Like reference numerals in the drawings denote like elements.

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art. Descriptions of well-known functions and structures are omitted to enhance clarity and conciseness.

It will be understood that when an element or layer is referred to as being “on” or “connected to” another element or layer, it can be directly on or directly connected to the other element or layer, or intervening elements or layers may be present. In contrast, when an element or layer is referred to as being “directly on” or “directly connected to” another element or layer, there are no intervening elements or layers present. It will be understood that for the purposes of this disclosure, “at least one of X, Y, and Z” can be construed as X only, Y only, Z only, or any combination of two or more items X, Y, and Z (e.g., XYZ, XY, YY, YZ, ZZ).

### Inflatable Portable Surgical Systems (Overall Configuration)

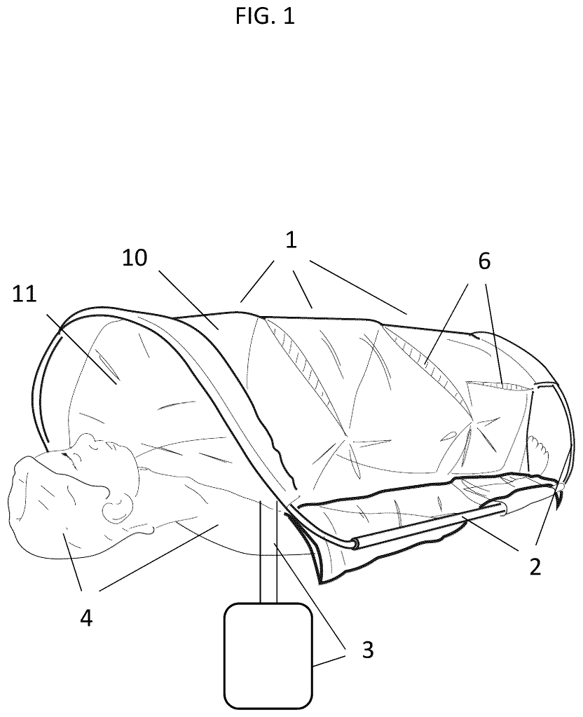

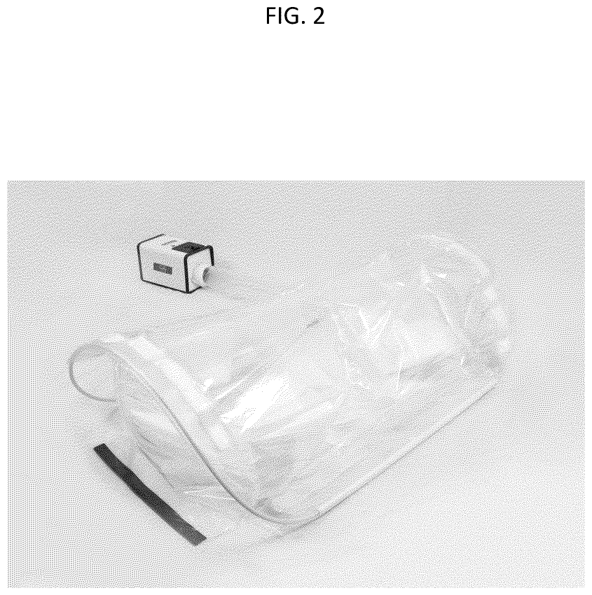

The configuration of an exemplary embodiment of a portable surgical system is described hereinafter with reference to FIGS. 1-3. The portable surgical system may include a flexible surgical enclosure 1, a frame 2, and an environmental control system 3.

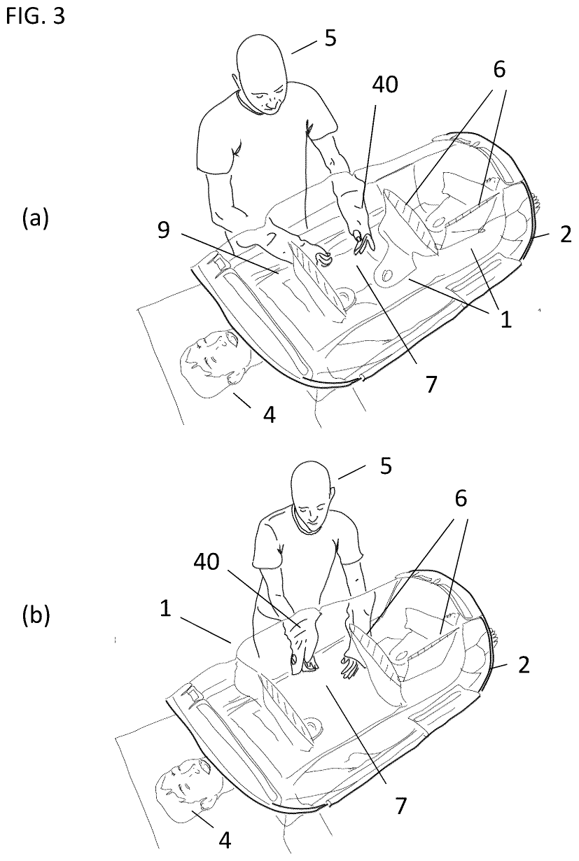

The surgical enclosure is configured to be disposed over the body of a patient 4 such that one or more operators 5 (e.g., a surgeon, a nurse, etc.) can access and perform a surgical procedure, from the inside of the enclosure, on a planned surgical-site 7 of the patient, such as on the abdomen, on the chest, on the back, etc. (see FIG. 3). The planned surgical-site may be referred hereinafter as the operating field. The surgical enclosure 1 is made, at least partially, of a transparent flexible material (i.e., transparent-material-layers) such that the operators can view the operating-field.

The enclosure may further include one or more incise drapes configured to be removed prior to performing the surgical procedure so that the user can access the operating-field. The enclosure may include an adhesive-surface configured to be adhered to the patient 4 so as to encompass the surgical-site 7 of the patient during the operation. The adhesive-surface of the enclosure may encompass the one or more incise-drapes of the enclosure so that, after the enclosure is attached to the patient, the one or more incise-drapes can be removed thereby exposing the surgical-site from the inside of the enclosure. This way the operators will be able to access and operate on the surgical site from the inside of the enclosure.

The surgical enclosure is configured to be supplied with air, via the environmental control system 3, so as to form an inner sterile space/environment enclosed by the enclosure above the operating-filed, thereby enabling the user (e.g., a surgeon) to perform surgery in a sterile environment. The surgical enclosure may be configured to be supplied with air under positive pressure. The portable surgical system may be configured such that filtered air is blown into the enclosure.

The enclosure 1 integrates arm ports 6 to allow access to the inside of the enclosure by either operator arms or augmenting instrumentation taking the place of arms such as laparoscopes or robots. Material ports which can be repeatedly opened and closed are used to maintain enclosure environmental integrity but allow the passing of anatomical specimens, instruments, and other materials into and out of the enclosure during a procedure. The surgical system may incorporate into the enclosure, and within proximity of the surgical-site, materials and instruments needed during the surgical procedure.

The enclosure may be attached to a frame 2 which is at least partially rigid. The frame is configured to provide support to the flexible surgical enclosure 1 and may cause the enclosure to assume a desired shape. The frame may be modular and may include rigid materials, such as plastic, rigid polyvinyl tubes, aluminum tubing, etc.

In an exemplary embodiment, the portable surgical system may not include a rigid frame such as frame 2. In an exemplary embodiment the portable surgical system may include one or more inflatable-beams or inflatable-structures configured to be inflated at a relatively high pressure so as to acquire relative rigidity and to provide shape and support to the enclosure. The inflatable-beams and inflatable-structures may be either incorporated into the flexible enclosure or may be attached to the enclosure.

The portable surgical system allows the operators to perform surgical procedures while keeping the surgical-site in sterile conditions by preventing contaminants from the environment and from the patient to reach the surgical site. At the same time the enclosure forms a barrier preventing biological materials generated during the surgical procedure (e.g., blood) from exiting the enclosure and reaching the operators, thereby protecting the operators.

In an exemplary embodiment the surgical enclosure may be single use disposable enclosure. In an exemplary embodiment, prior to the set-up/deployment for operation, the surgical enclosure may be supplied folded, like a surgical gown, and packed so as to be easy to store and carry on the field.

### The Surgical Enclosure

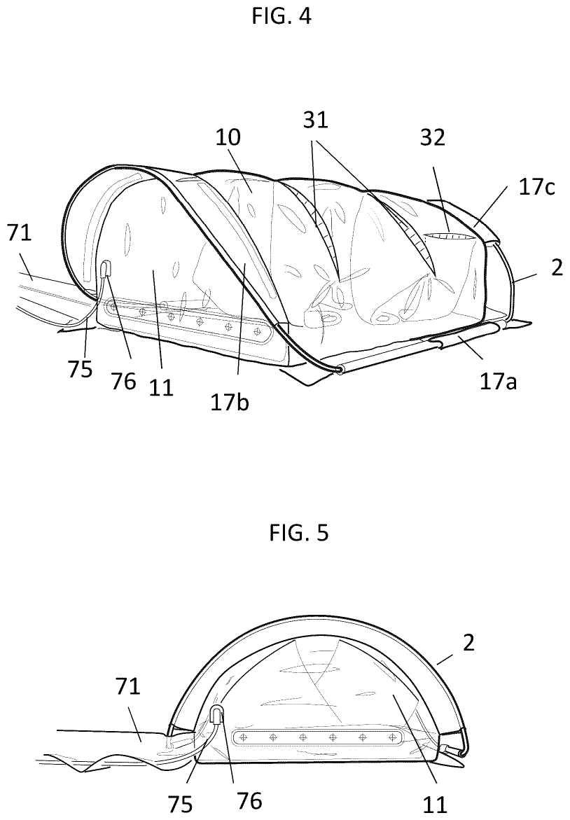

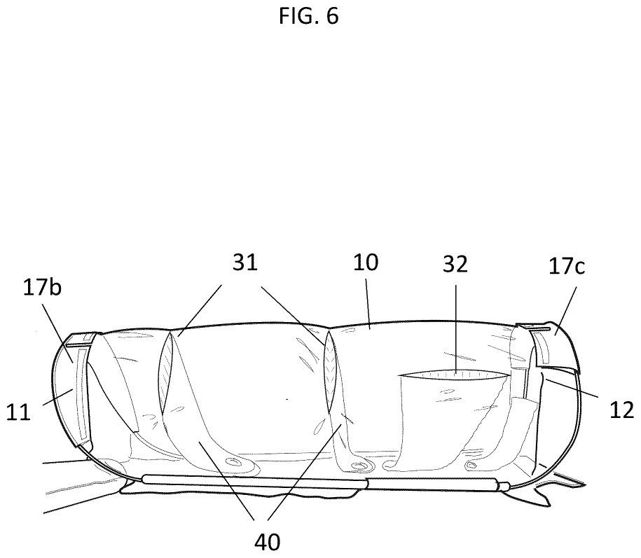

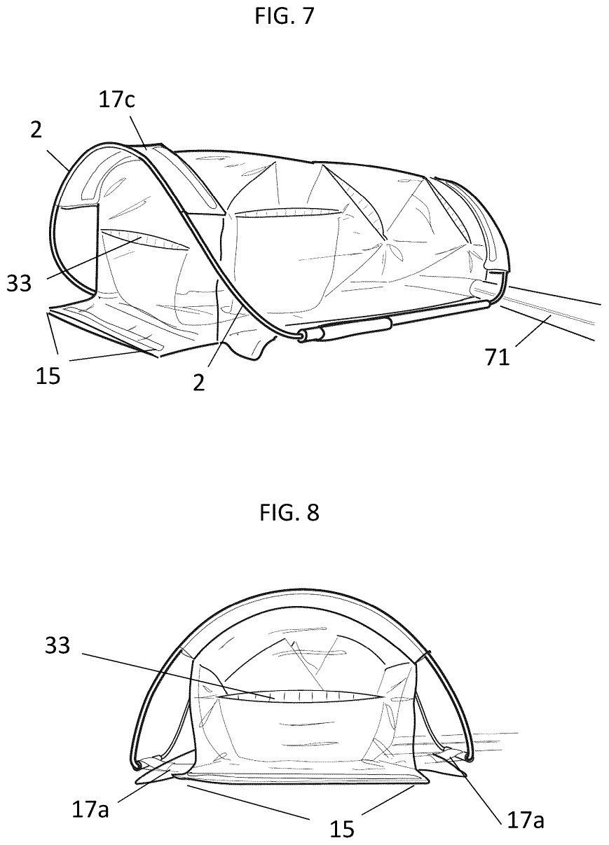

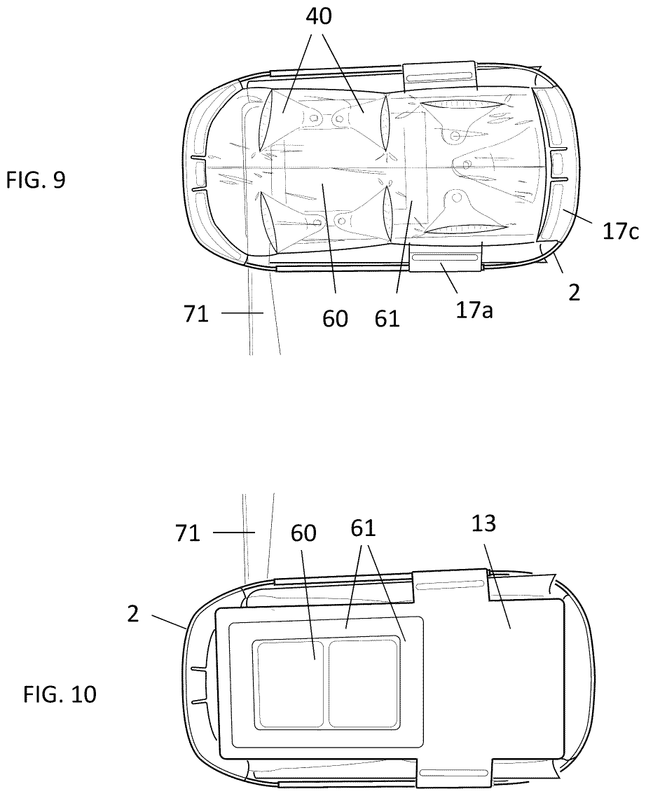

Various features and configurations of the surgical enclosure are described hereinafter with reference to FIGS. 4-10. FIG. 4 shows an oblique front-side perspective view of the frame and the surgical enclosure. FIG. 5 shows a front-side view of the frame and surgical enclosure. FIG. 6 shows a side view of the frame and surgical enclosure. FIG. 7 shows an oblique back-side view of the frame and surgical enclosure. FIG. 8 shows a back-side view of the frame and surgical enclosure. FIG. 9 shows a top side view of the frame and surgical enclosure. FIG. 10 shows a top view of the frame and surgical enclosure.

The enclosure may include a top-part 10 consisting of the enclosure which may have approximately a semi-cylindrical shape and may incorporate both a top and the sides of the enclosure.

The top-part may comprise one or more top and side view regions or panels of transparent enclosure material including optically-clear plastic, such as polyvinyl chloride and/or thermoplastic polyurethane (TPU), so as to permit the operators to view inside the enclosure. In an exemplary embodiment the transparent enclosure material may be a thermoplastic polyurethane (TPU) of about 2 mil, or 4 mil, or 6 mil, or 8 mil, or 10 mil, or 12 mil thickness, or higher or other values as may be appropriate from a manufacturability, ease of use, visibility, flexibility, or other desirable material properties known in the art. The transparent enclosure material may be configured to have one or more of the following qualities: good resilience, abrasion resistance, hydrolytic stability and resistance to attack by microorganisms; durability (for puncture, tear resistance); clarity (for optimal viewing); and stickiness.

The remainder of the surgical enclosure may comprise a flexible, impermeable plastic, such as low-density polyethylene and/or opaque TPU. In an exemplary embodiment the reminder of the surgical enclosure material may be an opaque thermoplastic polyurethane (TPU) of about 2 mil, or 4 mil, or 6 mil, or 8 mil, or 10 mil thickness, or any other material thickness reasonable for manufacturability, visibility, and flexibility of the enclosure. The transparent enclosure material may be configured to have one or more of the following qualities: good resilience, abrasion resistance, hydrolytic stability and resistance to attack by microorganisms; stickiness (e.g., extremely low stickiness to facilitate airflow and prevent kinking in the tube), and durability (for puncture, tear resistance).

The enclosure may include a front-side 11 disposed proximate to the head of the patient (see FIGS. 1, 4-6) and a back-side 12 disposed proximate to the feet of the patient (see FIGS. 6-8). The enclosure may further include a bottom-side 13 (see FIG. 10) disposed in contact with and attached to the body of the patient so as to allow access to the surgical site. The top-part 10, the front-part 11, the back-part 12, and the bottom-part 13 may be either formed from the same continuous sheet of material or may be formed from multiple sheets attached to each other via RF welding, heat welding, stitching, ultrasonic bonding, etc.

The enclosure may include a plurality of arm-ports 6 and sleeves 40 configured to enable operators to access the surgical-site. The surgical enclosure may further include one or more material ports configured to enable the moving of materials between the inside of the enclosure and the outside environment. The surgical enclosure may further include one or more line-ports configured to provide ongoing access for lines, tubes, wires, and drains requiring access to external resources (e.g., anesthesiology and breathing tubes, wires for medical devices, wires for sensors monitoring the patient).

### The Frame and Attachments to Enclosure

With reference to FIGS. 4-10, the surgical enclosure 1 is attached to the frame 2 via one or more attachment-means 17. The attachment means 17 may be disposed at a plurality of positions around the frame so as to achieve a desired attachment between the enclosure and the frame (see e.g. FIGS. 4-10). For example, attachment 17a may be disposed on the sides of the enclosure thereby attaching the enclosure with the lower sides of the frame (see e.g. FIGS. 8 and 9). Attachment 17b may be disposed at the front upper side of the enclosure thereby attaching the enclosure with the upper sides of the frame (see e.g. FIGS. 4 and 6). Attachment 17c may be disposed at the back upper side of the enclosure thereby attaching the enclosure to the upper side of the frame (see e.g. FIG. 4, 6, 7).

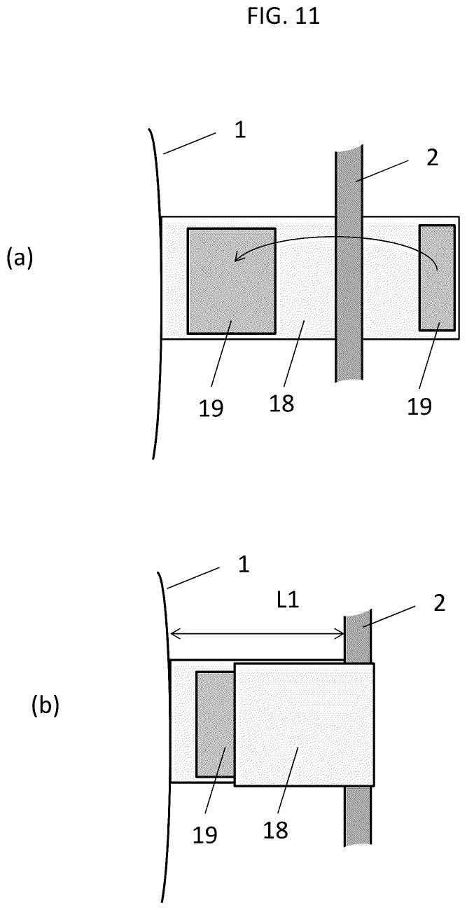

With reference to FIG. 11(a), the attachment-means may include a material-slab 18 attached (e.g., stitched, or welded) to the lower part of the enclosure and one or more Velcro pads 19 attached on the material slab 18. FIG. 11(a ) shows a configuration where the attachment-means is disconnected from the frame 2. FIG. 11(b) shows a configuration where the material-slab 18 is wrapped around a portion of the frame 2 and the Velcro pads 19 attach to each other thereby attaching the enclosure 1 to the portion of the frame 2. The distance between the frame and the enclosure may be adjusted to a desired length “L1” by adjusting the position of the Velcro pads with respect to each other.

Whereas for attachment 17a the frame portion has a straight cylindrical shape and the slab may conform neatly following the shape of the frame, the frame portion for attachments 17b and 17c may have a bent cylindrical shape on which a rectangular slab does not conform. The attachments 17b and 17c may be designed such as to conform to the bent shape of the frame in the upper front and back sides of the frame. It will be understood that various other attaching means may be used without changing the spirit of the invention.

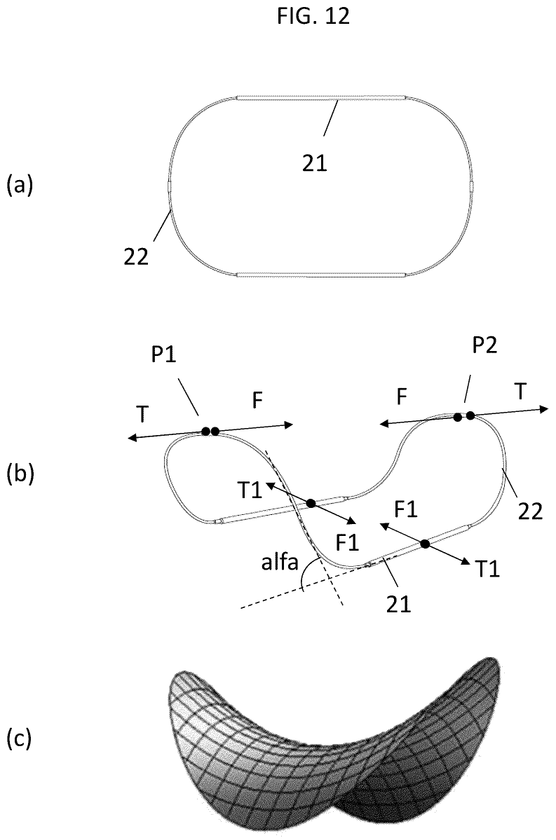

FIG. 12 show an exemplary embodiment of a frame 2. The frame 2 may include spacer-sections 21 and tensioner-sections 22 connected with each other so as to form a closed loop. The spacer-sections 21 are essentially rigid (do not change shape) whereas the tensioner sections are configured to change shape when outside forces are applied and provide spring-like resistance/forces. When no constraints or outside forces are applied on frame 2, the frame takes the planar state/form shown in FIG. 12(a). When outside forces “F” are applied on the tensioners 22 and forces “F1” are applied on the spacers 21 the loop may bend and assume a saddle shape as shown in FIG. 12(b). The forces “F” determine the angle “alfa” formed by the tensioner section with the planar surface of the spacer-sections 22. The force F1 determines the spacing between the spacers 21 and the arc of the tensioners 22. Conversely, the spring-like frame material of the tensioners 22, tensioned into the bent shape of FIG. 12(b) is configured to generate tension forces “T” and “T1” opposite to the forces “F” and “F1”.

In an exemplary embodiment of the invention each tensioner-segment of the frame may assume, substantially and approximately, the shape formed at the intersection between a hyperbolic paraboloid surface (such as the surface in FIG. 12(c)) and a half-section of a cylindrical surface having an elliptical cross-section. The two tensioners 22, each constituting half of a saddle shape, are spaced apart by the two spacers 21, thereby forming an elongated saddle shape. In mathematical terms the tensioner-segments may substantially follow a line satisfying the following equations:

$$\\ \begin{matrix}z = \frac{{x}^{2}}{{a}^{2}} - \frac{{y}^{2}}{{b}^{2}} ; & ( equation 1 )\end{matrix}$$

$$\\ \begin{matrix}\frac{{x}^{2}}{{c}^{2}} + \frac{{y}^{2}}{{d}^{2}} = 1 ; & ( equation 2 )\end{matrix}$$

$$\\ \begin{matrix}x > 0 & ( equation 3 )\end{matrix}$$

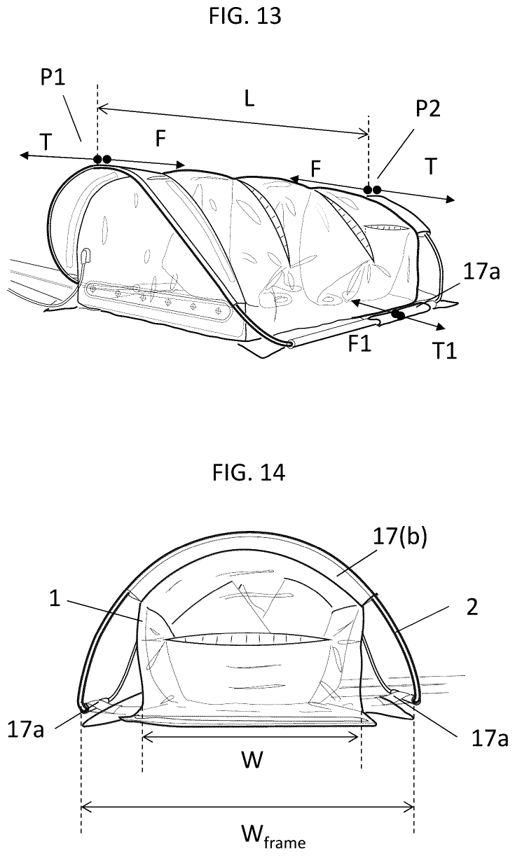

With reference to FIG. 13, in an exemplary embodiment a mid-point P1 of the first tensioner-segment is attached to a front axial-end of the enclosure and a mid-point P2 of the second tensioner-segment is attached to a back-axial-end of the enclosure. The tension over the axial length of the enclosure is generated by the tensions in the bent tensioner-segments. When the surgical-enclosure 1 is attached to the frame 2, at P1 and P2, via at least attachment—means 17b and 17c, the tensions “T” generated in frame 2 bent into the elongated saddle shape are used to stretch the enclosure to its axial length “L” and into the desired volume and shape. The surgical-enclosure 1 is configured such that the axial length “L” of the top material of the enclosure (including the width of attachments 17b and 17c) is approximately equal to the length between the two top saddle points P1 and P2 of the frame shape. The enclosure axial length “L” imposes length constraints on the shape of the frame 2. In other words, whereas the enclosure material 1 acts with forces “F” upon the frame 2 thereby keeping the frame into its saddle shape, the frame 2 acts with forces T upon the surgical enclosure 1 thereby stretching the enclosure to its desired axial length and shape. A similar tension-constraint relationship occurs between the frame 2 and the enclosure 1 via attachment points 17a: the enclosure exerts a force F1 on the frame 2 whereas frame 2 exerts T1 reaction force on the enclosure.

It has been determined by the inventors herein that a tensioned saddle shape frame as described above provides an optimal shape to the surgical enclosure which translates into optimal operating conditions for the operators. This configuration allows for designing tensioned saddle shaped frames which are light-weight and portable (the frame uses reciprocal tension-constraint forces applied via spring constant rather than an otherwise necessary rigid frame).

With reference to FIG. 14, the lower part of the frame 2 may be connected to the surgical enclosure via attachment-means 17c thereby stretching the enclosure along its width. The width “W” of the surgical-enclosure and the stretch of the bottom side of the enclosure may be adjusted by adjusting the length of the attachment-means 17c.

The shape of the flexible surgical enclosure 1 (e.g. configurations and distances between various parts of the flexible surgical enclosure) may be controlled via attachment-means such as 17. Multiple attachment-means may connect various sections of the flexible surgical enclosure 1 with various sections of the frame 2 such as to provide the desired form and shape of the enclosure. The shape of the surgical enclosure and tensions in the enclosure material may be further adjusted by adjusting the length of the attachment-means 17.

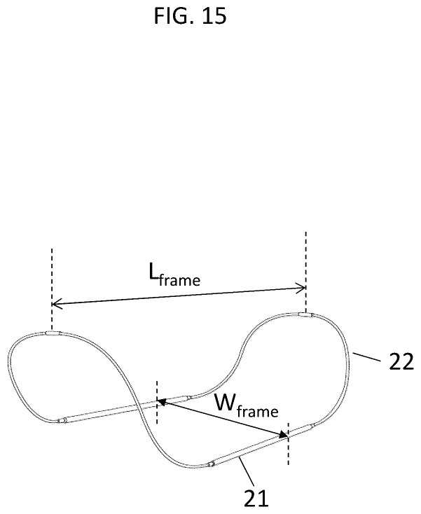

In an exemplary embodiment, the frame length “Lframe” and frame width “Wframe” (see FIG. 15) may be adjustable by providing spacer-sections and tensioner-sections of adjustable length. The size, volume and shape of the flexible enclosure may be adjusted by adjusting the frame length “Lframe” and the frame width “Wframe”. Similarly, the slack and tensions in certain portions of the enclosure material may be adjusted by adjusting the frame length “Lframe” and frame width “Wframe”.

In an exemplary embodiment, the shape, volume and slack/tension in certain portions of flexible enclosure may be adjustable so as to fit patients of different sizes and different anatomical structures. For example, in the case of an adult patient having a broader than average chest, the width and/or slack of the bottom-side 13 may be adjusted (e.g., by adjusting the frame width and/or the length of the attachment means 17) so as to fit the chest. In the case of a young patient such as a child, the width and/or slack of the bottom-side 13 may be adjusted down (e.g., by adjusting the frame width and/or the length of the attachment means 17) such as to fit the patient.

In an exemplary embodiment, the bottom-side of the enclosure may include a material-fold 18 which may be deployed such as to provide different widths for the bottom-side 13 (see FIG. 15). When in a completely unfolded-state the bottom-side width is maximum Wmax. When in a completely folded-state the bottom-side width is minimum Wmin. Intermediate states of folding provide intermediate width for the bottom-side. Similar folds may be provided at various positions and on different parts of the enclosure (e.g., top-side 10, front-side-11, back-side 12) thereby providing a means for adjusting the volume, shape, and various other dimensions of the enclosure according to the operating/procedural needs.

### The Modular Frame

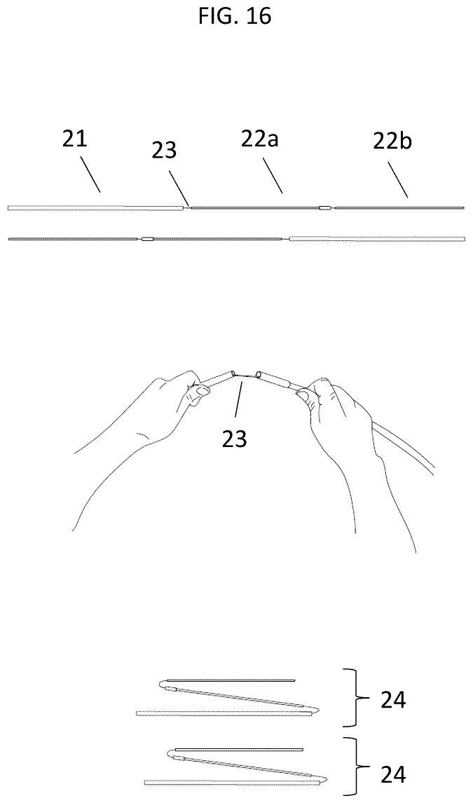

With reference to FIG. 16, in an exemplary embodiment the frame 2 may include several modular segments configured to be assembled into the frame 2. For example, the frame may include the spacer 21 and two tensioner sections 22a and 22b linked to each other via strings 23. The frame segments may be linked to each other into two sections 24 via strings 23. When assembled the sections 22a and 22b form the tensioner 22. The frame 2 may be formed by connecting the segments into sections 24 followed by connecting the two sections 24 and bending sections 22 so as to form the frame in FIG. 12(a).

### The Inflatable-structure-frame

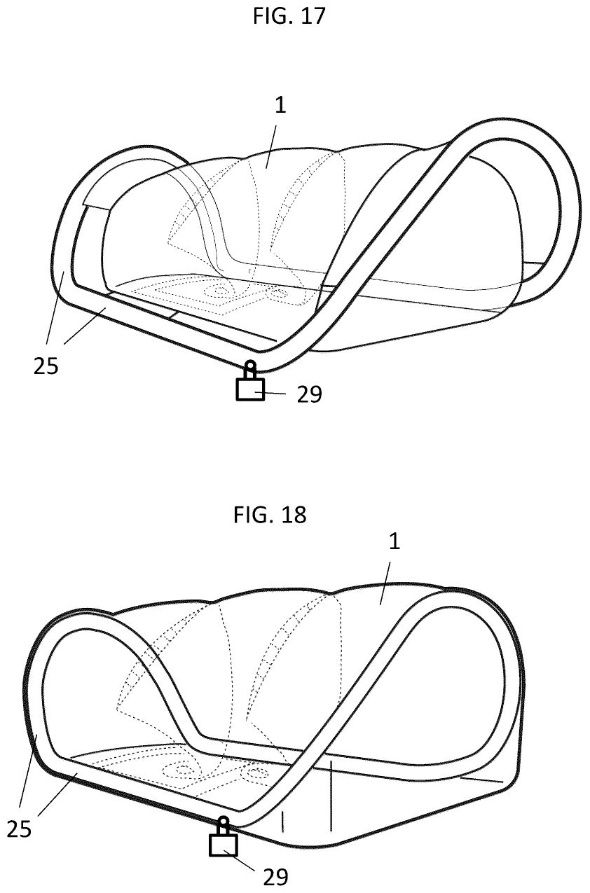

As described hereinafter with reference to FIGS. 17-20, exemplary embodiments of the portable surgical system may include one or more inflatable-structures 25 (instead of frames made of rigid or spring like materials, such as frame 2) configured to be inflated at a relatively high pressure so as to acquire relative rigidity and to provide shape and support to the enclosure. The inflatable-structures 25 may be made of flexible materials (e.g. the same material as the enclosure material or a thicker material, polyethylene, plastic sheet, polymer films, woven textiles, laminated textiles, non-woven textiles, etc.) and may be air-tight. Such inflatable structure may be single layered or multi-layered with an inner layer creating an airtight bladder and an outer layer patterned into a predetermined shape. The inflatable-structures 25 may be either incorporated into the flexible enclosure or may be attached to the enclosure.

The inflatable-structure 25 may further include an inflation-port. An air/gas-source 29 (e.g., compressed gas cartridge, pump) may be attached to the inflatable-structure 25 via the inflation-port and may provide pressurized gas (e.g., CO2, Nitrogen, compressed air) to the inflatable-structure so as to create a relatively high pressure into the inflatable-structures. The inflatable-structures 25 may be configured to be inflated at significant higher pressures than the pressure inside the surgical enclosure 1. The inflatable-structures 25 may be made of flexible materials withstanding higher pressures than the enclosure material and more resistant to breaking (e.g., thicker plastic/polymer layers or textile layers). The inflatable-structures material may be a transparent material so as not to obstruct viewing inside the enclosure.

The gas source 29 may include a compressed gas cartridge or canister including pressurized gas such as CO2. The gas source 29 may provide pressurized gas generated via a chemical reaction between two or several compounds included in a container. Such a container could be attached directly to the frame and include multiple nesting containers which are designed to be rupturable and together comprise a compression-triggered mechanism to initiate a chemical reaction resulting in inflation. The gas source 29 may include an external air or gas pump. The gas source 29 may include a trigger-device configured to trigger the release of pressurized gas into the inflatable-structures 25 thereby autonomously and quickly inflating the inflatable-structures. The gas cartridge is configured to inflate the inflatable-structure to a desired inflatable-structure-pressure upon the activation of a trigger-device. The gas source 29 may include one or more pressure control devices for ensuring that appropriate pressure is created in the inflatable-structures 25 and for preventing overpressure in the inflatable-structures (e.g., pressure gauges, overpressure valves, regulators, shut-off valves). Pressurized gas cartridges have the advantage that they are small, light, easy to use, provide quick inflation at the desired pressure to the inflatable-structure.

FIG. 17 shows an exemplary embodiment including an inflatable-structure 25 having a saddle. When in an inflated state (such as when the surgical system is in use), the shape of the inflatable-structure 25 may be substantially the same or similar to the shape of the frame 2 described with reference to FIGS. 1-16). The inflatable-structure may be disposed outside the enclosure and may be attached to the material of the enclosure (e.g., around peripheral edges of the enclosure) such as to provide shape and structure to the enclosure. The inflatable-structure may be attached to the enclosure via attachment means such as 17. The inflatable-structure may be directly incorporated into the enclosure via attachment means such as stitching or heat/RF welding along edges of the enclosure 1 and edges of the inflatable structures. When in an inflated state, the inflatable-structure are configured to acquire relative rigidity and to provide shape and support to the enclosure.

FIG. 18 shows an exemplary embodiment of a portable surgical system including an inflatable-structure 25 disposed substantially inside the enclosure 1. The inflatable-structure 25 may be incorporated into part of the enclosure and may be attached to the enclosure material. The inflatable-structure 25 may have a saddle shape (e.g., as shown in FIG. 18) or various other shapes.

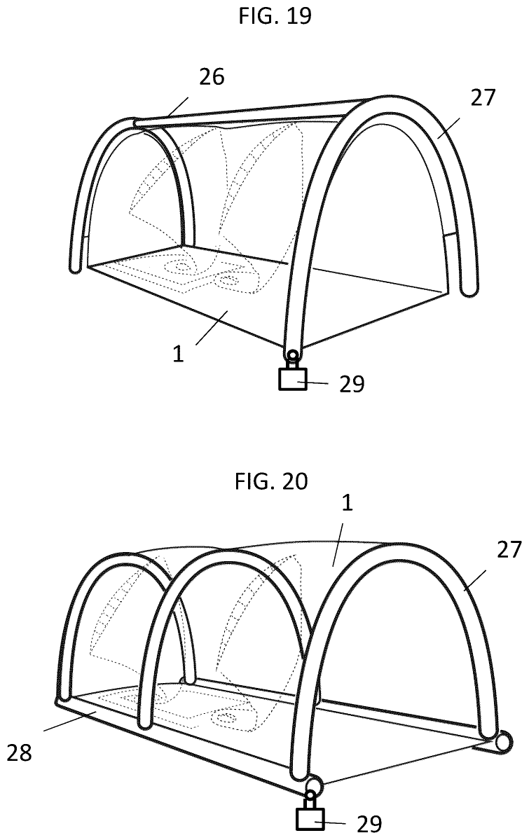

FIG. 19 shows an exemplary embodiment for which the inflatable-structure 25 includes a top-air-beam 26 and two rib-air-beams 27. The top-air-beam 26 may be disposed axially over the enclosure 1 whereas the rib-air-beams may be disposed at and attached to the back and front of the enclosure 1 (as shown in FIG. 19). The inflatable-structure 25 may be incorporated into part of the enclosure and/or may be attached to the enclosure 1.

FIG. 20 shows an exemplary embodiment for which the inflatable-structure 25 includes two base-air-beams 28 and three rib-air-beams 27. The two base-air-beams 28 may be disposed axially along the base of the enclosure 1 whereas the rib-air-beams may be disposed at and attached to the back, middle, and front of the enclosure 1 (as shown in FIG. 20). The inflatable-structure 25 may be attached to the enclosure 1 and may be incorporated into part of the enclosure.

In a deflated state the inflatable-structures 25 may collapse into a foldable flexible structure. As previously mentioned, prior to the set-up/deployment for operation the surgical enclosure 1 may be folded like a surgical gown. In the folded state, the inflatable-structure 25 may be folded together with the enclosure 1. Upon inflation of the inflatable-structure 25 at the desired pressure the inflatable-structure assumes the desired shape (e.g., saddle) and stretches the enclosure into the desired expanded operating shape for performing surgical procedures. The inflatable-structure will provide support to the walls of the enclosure and reinforce the enclosure into the desired shape.

The inventions herein are not limited by the particular shapes and configuration of the inflatable-structures. The skilled artisan would understand that various shapes, configurations and materials may be employed and are within the scope of the inventions.

### The Arm Ports and the Sleeves

The surgical-enclosure 1 may include a plurality of arm-ports 6 enabling the operators to access the surgical site from the inside of the enclosure as seen in FIGS. 1-10. In an exemplary embodiment the surgical enclosure may include several arm ports (e.g., 31 and 32 in FIGS. 4 and 6) on each side of the top-part of the enclosure. The arm-ports may be formed by cutting the enclosure material along straight or angled lines. For example, arm-ports 31 are formed by cutting the enclosure material along straight lines perpendicular to the axis of the enclosure whereas arm-ports 32 are formed by cutting the enclosure via straight lines parallel with the axis of the enclosure.

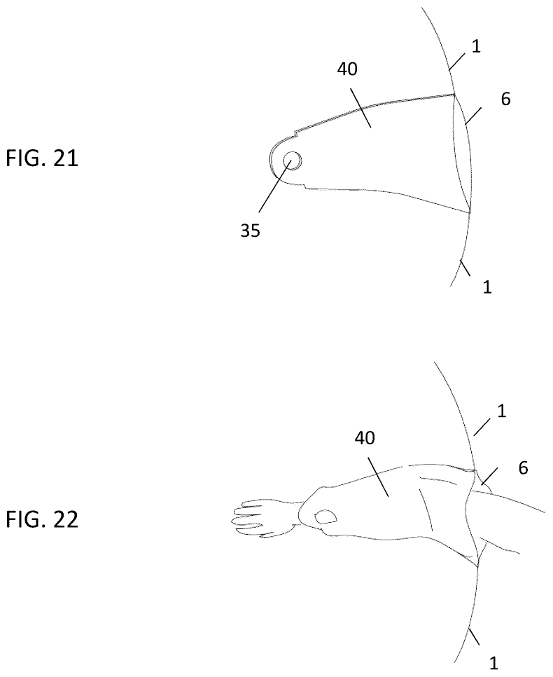

The enclosure may further include a plurality of sleeves 40 enabling the operator to access and operate on the surgical-site (see FIGS. 3, 6, 9). The sleeves may be connected to the arm-ports (as shown in FIGS. 21 and 22) by various means such as stitching, heat welding, RF welding, ultrasonic bonding. The sleeves are configured to accommodate an arm of the operator to perform work on the surgical site. The sleeve may further include a means for securing the sleeve on the hand or arms of the operator, such as: a strap, an elastic band, a string, a thread, holes in the material, etc. In an exemplary embodiment of the invention some of the sleeves may include a first hole 35 on a side of the sleeve so as to accommodate a thumb of the right arm and a second hole 36 so as to accommodate a left arm thumb (as seen in FIG. 22). The arm ports and the sleeves may include means (e.g., a strap, an elastic band, a string, etc.) for sealing the sleeve material or other materials on the arm of the patient so as to prevent fluids from moving between the inside and outside of the enclosure via the sleeve and ports.

It is understood that during surgery only some of the sleeves may be used by the operator(s) while some sleeves may not be used. The sleeves which are not in use during surgery may be folded and disposed (or attached) on the side of the enclosure such that the folded sleeves do not block the view of the surgical site, do not get in the way of the operators, and do not allow air flow through the sleeves between the inside and outside of the enclosure.

The material of the sleeves may be a two sided material: an inner side of the sleeve facing the arm and hand of the operator while in use by the operator; and an outer side of the sleeve facing towards the enclosure environment. The inner side of the sleeve may be configured to be comfortable on touch (e.g., soft, wicks up moisture). The outer side of the sleeve may be configured to be impermeable to fluids such as blood. The material of the sleeve may be a polyurethane laminate Spun Bonded Nonwoven. The sleeve material may have a thickness of about 2 mil, 4 mil, 6 mil, 8 mil, 10 mil, or other standard material thicknesses, as may be found appropriate for ease of use, comfort of operator, or manufacturability. The sleeve material may be a waterproof medical fabric. The sleeve material may be configured to have one or more of the following qualities: comfort; lack of permeability so as to prevent air/water from transferring between the patient and practitioner); and ease of attachment to the material of the enclosure).

### The Patient-limb-port

The surgical enclosure may include one or more ports 33 disposed on the back-side 12 of the enclosure 1, as seen in FIGS. 7, 8, 18, and 19. The port 33 may be used as arm-port thereby enabling an operator to access the enclosure from the back-side. In an exemplary embodiment, the port 33 may be used to perform surgery on an arm, hand, leg or foot of a patient. The port 33 may be referred hereinafter as a patient-limb-port whereas the surgical site on a limb may be referred as limb-surgical-site.

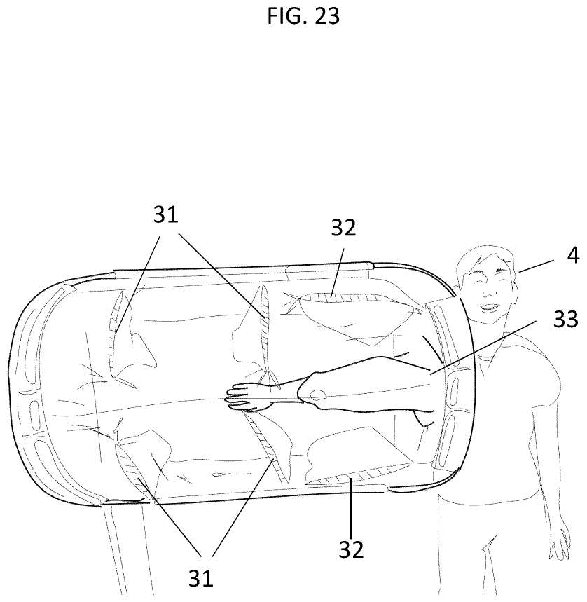

With reference to FIG. 23, a patient 4 may lay on his back on the ground or some other surface. The surgical-system may be disposed next to the patient such that the patient can insert into the surgical enclosure an arm (to be operated on via) the port 33 disposed on the back side of the surgical enclosure 1. One or more operators may perform surgery on the patient's arm or hand via the ports 31 and 32. The port 33 may include means (e.g. a strap, an elastic band , a string, etc.) for sealing a sleeve material or other materials on the arm of the patient so as to prevent fluids from moving between the inside and outside of the enclosure via the sleeve and ports.

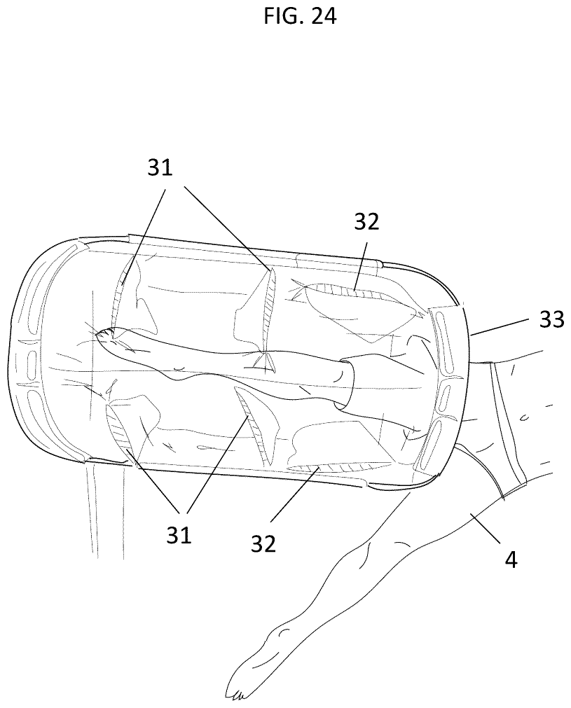

With reference to FIG. 24, a patient 4 may lay on his back on the ground or some other surface. The surgical-system may be disposed next to the patient such that the patient can insert into the surgical enclosure a leg via the port 33 disposed on the back side of the surgical enclosure 1. One or more operators may perform surgery on the patient's leg or foot via the ports 31 and 32. The port 33 may be made to be of adjustable size such as to accommodate different leg and arm sizes.

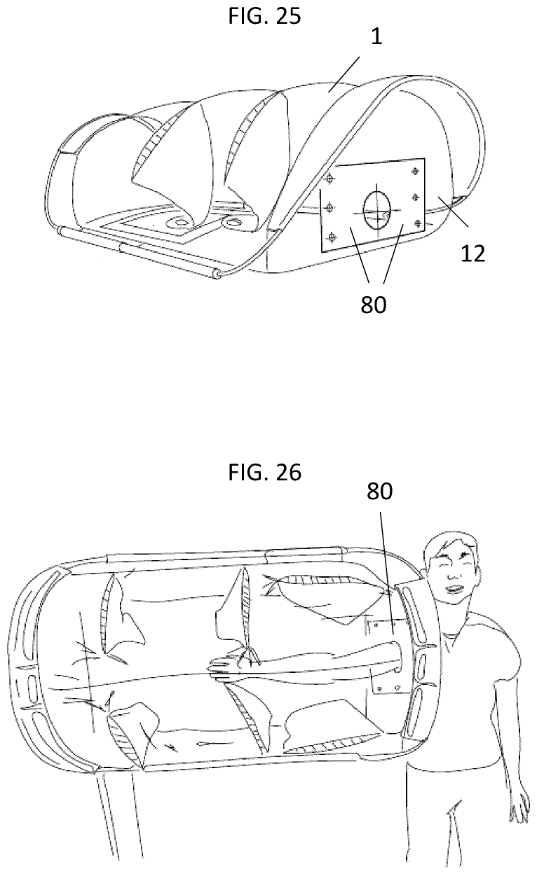

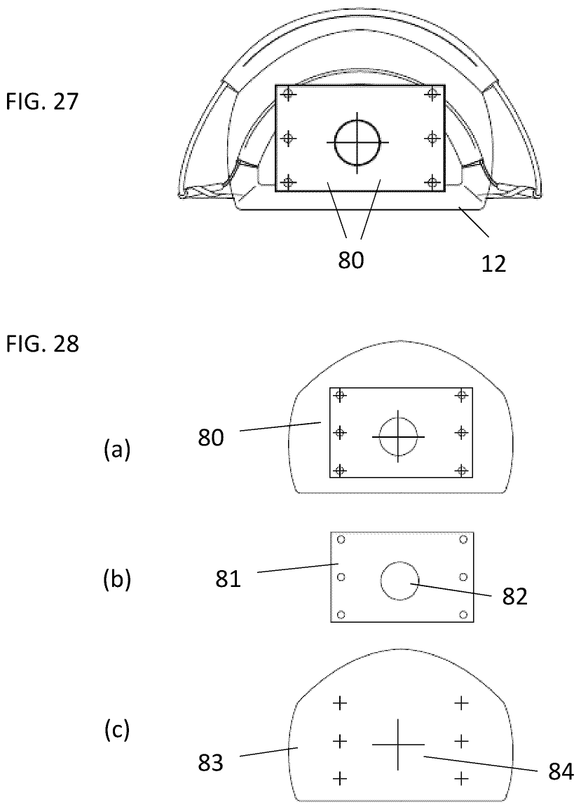

FIGS. 25-27 show an alternate embodiment of the surgical system where the arm and leg port disposed on the back-side 12 of the enclosure 1 (e.g. port 33 in FIGS. 8, 23, 24) is a two-layer port 80. As seen in FIG. 26, the two-layer port 80 may be used to perform surgery on an arm, hand, leg or foot of a patient. Port 80 may also be used as arm-port thereby enabling an operator to access the enclosure from the back-side.

With reference to FIGS. 28(a)-(c), the two-layer port 80 may include a bottom-layer 81 (as seen in FIG. 28(b)) and a top-layer 83 (as seen in FIG. 28(c)). The top-layer 83 has a cross-cut-pattern 84 which may be airtight while including a fine cut pattern enabling an operator to break the fine cut-pattern along the pattern lines. The bottom-layer 81 includes a cut hole 82 which may be of circular shape. The top-layer 83 may be disposed over the bottom-layer 81 and the bottom-layer may be attached or incorporated into the back-side 12 of enclosure 1. The cross-cut pattern 84 may be substantially centered with the hole 82. The diameter of the cut hole 82 may be smaller than the lengths of the cross-cut lines of the cross-cut pattern 84. The top-layer 83 and bottom layer 81 may be made of flexible plastic material layers (e.g. thermoplastic polyurethane (TPU), polyethylene, polyvinyl chloride, the same material as the enclosure material, etc.). The bottom-layer 81 may be made of a stretchier and/or thicker material than the top-layer 83 (e.g., a different mil and type of TPU may be used).

Prior to use the two-layer port 80 may be substantially airtight sealed since the fine cut pattern 84 is airtight. The two-layer port 80 may be opened during operation by breaking the cross-cut pattern 84 along the fine cut pattern. An arm or a leg may be inserted into the enclosure 1 through the broken cross-cut-pattern 84 of the top-layer 83 and the hole 82 of the bottom-layer 81 (as seen in FIG. 26).

### The Material Ports

The surgical enclosure may further include one or more material ports 15 configured to enable the moving of materials and instruments between the inside of the enclosure and the outside environment (as seen in FIGS. 7, 8 and 29). For example, a material port 15 may be disposed on the back-side 12 of the enclosure. The material port 15 may be linear (e.g., created by forming a linear cut on the enclosure material) and may include two magnetic strips arranged on each of the two sides of the port so that when the two magnetic strips are in contact disposed over/against each other the port is in a closed state whereas when the strips are disconnected the port is open. The port may be opened/closed by connecting and disconnecting the two magnetic strips.

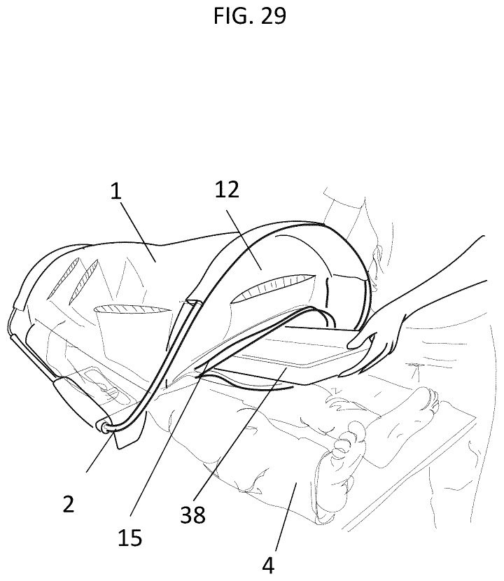

With reference to FIG. 29, the material port may be configured such that an instrument tray 38 can me moved in and out of the enclosure. The size of the material port may be configured such as to enable the moving of patient material, larger devices, instruments and trays.

### The Line Ports

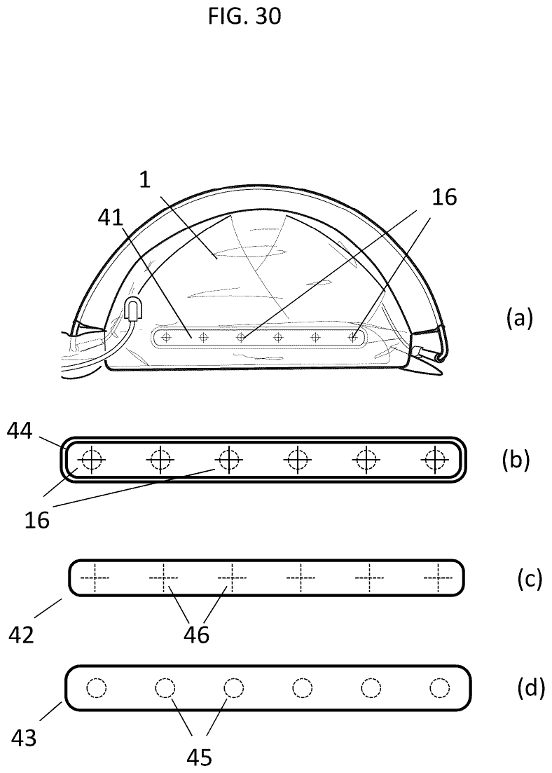

The surgical enclosure may further include and one or more line-ports 16 configured to provide ongoing access for lines, tubes, wires, and drains of medical devices requiring access to external resources (e.g., anesthesiology and breathing tubes, wires for medical devices, wires for sensors monitoring the patient). As seen in FIG. 30(a), a plurality of line ports 16 may be disposed on the front-side of the enclosure and may be arranged in a line port assembly 41. FIG. 30(b) shows an exemplary embodiment of a line port assembly 41 including six line ports 16. The line port assembly may include a first-layer 42 (shown in FIG. 30(c)) and a second-layer 43 (shown in FIG. 30(d)) disposed essentially on top of which other and in contact with each other. The first-layer and the second-layer may be connected (e.g. stitched, RF welded, heat welded, ultrasonically bonded) around the edges 44.

The first-layer 42 may include a series of circular-perforations 45. The second-layer 43 may include a series of cross-perforations 46 disposed essentially over the circular-regions 45, as shown in FIG. 30(b). A line-port may be formed by opening a circular perforation and its corresponding cross perforation. Either the first-layer 42 or the second-layer 43 may be contiguous with the enclosure material or may be part of the enclosure material.

Various lines (e.g., electricity wires, tubing, incubation lines, anesthesia lines, etc.) may be inserted into the enclosure from outside by, for example, penetrating/opening a circular perforation and its corresponding cross perforation. The line-ports 16 provide an easy and efficient way to insert tubes, lines, wires into the enclosure. At the same time the line-ports are ensuring a sufficiently tight seal between the lines/tubes and the layer materials 42-43 such as to provide the required barrier between the inner and outer environments and to ensure the required air sealing.

### The Fluids Reservoirs

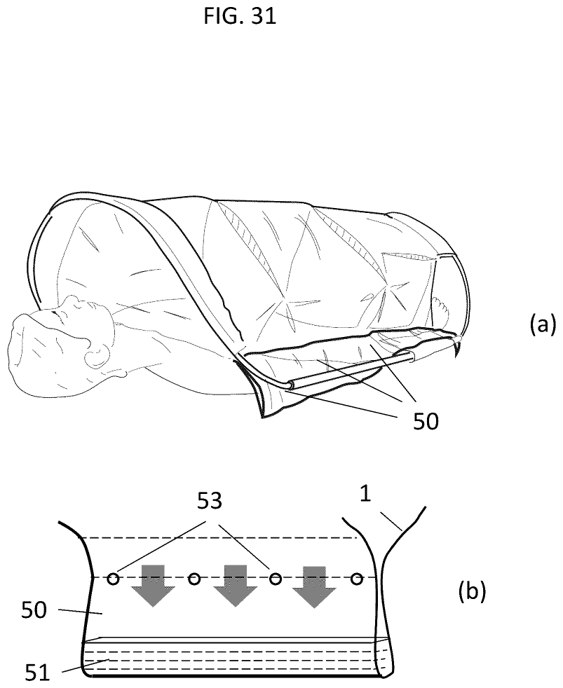

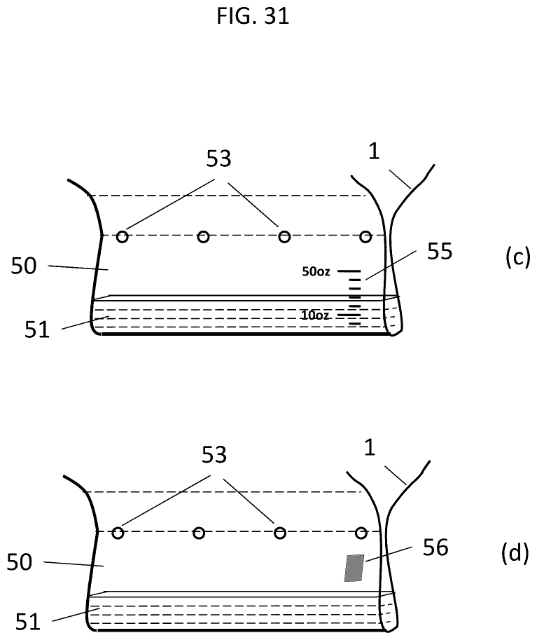

In exemplary embodiments of the invention, the surgical enclosure may include one or more fluids reservoirs 50, as described with reference to FIGS. 31(a)-(b). The fluids reservoir 50 may be disposed in the lower part of the enclosure so as to collect unwanted fluids 51, such as blood, generated inside the enclosure during the surgery. The fluids reservoir may be formed as a fold or pocket of material disposed on the lower part and on the side of the surgical enclosure. The fluids reservoir is connected with the enclosure so that the fluids generated into the enclosure drain into the reservoir.

The fluids reservoir may be made as a pocket or fold of the transparent enclosure material (e.g., they may be made from the same sheet as the transparent enclosure) so that the operators can view how much blood/fluids have been accumulated during the surgical procedure. FIG. 31(b) shows a section of the reservoir 50 made as a fold of the transparent material of the enclosure wherein the fold is created by welding the two parts of the fold at several points 53. The welding points 53 will create the pocket/fold of the reservoir while allowing the fluids to move from the inside of the surgical enclosure into the pocket in the regions between the welding points 53, as shown by the arrows in FIG. 31(b).

With reference to FIG. 31(c), in an exemplary embodiment the fluids reservoirs 50 may include scales 55 painted on the side of the reservoirs indicating the amount of fluids 51 (e.g. blood) collected in the reservoir.

With reference to FIG. 31(d), in an exemplary embodiment the fluids reservoirs may include a strain-sensor (such as “foil strain gauges” and other gauges/sensors as well known in the art) attached to the material of the reservoir and configured to measure the strain in the reservoir material. The accumulation of fluids 51 into the reservoir generates strain into the reservoir material which is measured by the strain-sensor. The measured strain is proportional/commensurate with the quantity of accumulated fluids 51. A device, such as a computer, may be configured to receive strain measurements from the strain sensor, to calculate the amount of fluids in the reservoir, and to display the amount of fluids on a monitor. This way operators are able to monitor the amount of fluids (e.g. blood) accumulated in the fluids-reservoirs 50.

### The Incise Drape

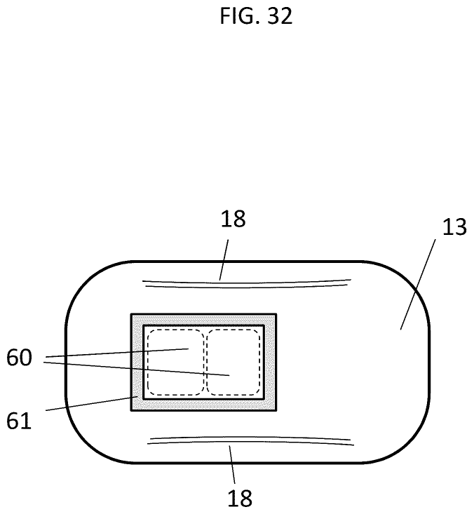

With reference to FIG. 32 and FIG. 10. The enclosure may further include one or more surgical incise drapes 60 incorporated into the bottom-part 13 of the enclosure. The bottom of the enclosure may further include an adhesive-surface 61 configured to be adhered to the patient so as to encompass the surgical-site of the patient during the operation. The adhesive-surface of the enclosure may encompass the one or more incise drapes of the enclosure so that, after the enclosure is attached to the patient, the one or more drapes can be removed thereby exposing the surgical-site from the inside of the enclosure. The incise drapes may be connected with the bottom via a perforated periphery line enabling the removal of the incise drapes. Removal of the incise drapes creates an opening into the enclosure over the surgical site of the patient. Thus the operators can perform surgery on the surgical site from the inside of the enclosure and through the opening created by removal of the incise drape.

The incise drape serves as the interface with the patient body. The size and shape of the incise drapes 60 may be configured to cover the surgical-site on the patient's body (e.g. the torso or the back) while essentially excluding body surface outside the surgical site. The surgical site on the torso may be referred hereinafter as a torso-surgical-site. Consequently, only the surgical site of the patient's body (i.e., area covered by the incise drape 60) is included within the surgical enclosure, while the remainder of the patient body is excluded from the surgical field (which may be kept as sterile as feasible). By excluding from the surgical enclosure the unnecessary body surface, the efficacy of the system is significantly improved since the patient's body surface contributes to environment contamination inside the enclosure. In particular, the exclusion of high-contaminant regions such as the oropharynx or the genitals is likely to significantly improve the efficacy of the system. The surgical enclosure may include one or more incise drapes of different shapes and sizes and may be disposed at different positions on the surgical enclosure such as to fit the needs of different types of medical procedures. The bottom of the surgical enclosure may include straps for securing the enclosure to the patient or to the operating table for additional stability.

### The Environmental Control System

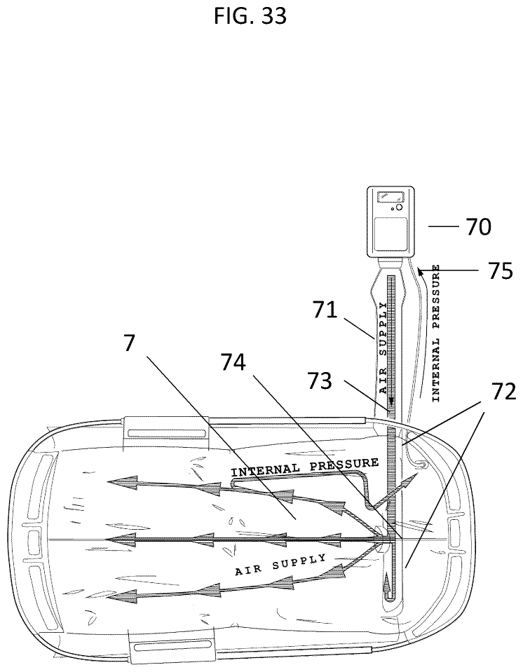

FIG. 33 shows schematically a bottom view of the surgical system on which the configuration and functioning of the environmental control system 3 is described. The environmental control system may include an external air-supply system, an internal air-supply system, and a pressure sensing system.

The external-air-supply system may include a fan, a battery, an air filter (e.g., HEPA filter), a control-system, a connector-tube 71. The fan, the battery and the control-system may be incorporated into a control-device 70. The internal-air-supply system may include an air-tube 72 including an air-inlet 73 configured be connected to the connector-tube 71. The tube 72 may be disposed on the bottom of the enclosure in the proximity of the front end and may include one or more air-outlets 74 positioned such as to supply air-flow to the desired areas of the enclosure. During operation the air-supplied by the fan is directed through the connector-tube 71 into the tube 72, via inlet 73, and further into the surgical enclosure via the air-outlets 74. The air-outlets 74 may be disposed such as to direct air-flow over the surgical site 7. As seen in FIG. 33, in an exemplary embodiment an air-outlet 74 is disposed approximately on the bottom axis and is configured to direct air-flow from the front side towards the back side and over the surgical site, as shown by the arrows. The air-tube 72 is disposed approximately perpendicular to the axis and proximate to the front side of the surgical enclosure.

The pressure sensing system may include a pressure sensor (which may be disposed in the control-device 70) and a pressure-tube 75 connected to the enclosure via connector 76 so as to allow air pressure from the enclosure to be measured by the pressure sensor (see FIGS. 4-5). The control-system is configured to control the pressure sensor and to receive the measured pressures from the pressure-sensor. The control-system is further configured to control the air-supply to the enclosure, function of the received pressure readings from the sensor, so as to provide the desired air-pressure inside the surgical enclosure. In an exemplary embodiment the control-system is configured to keep positive pressure (i.e., pressure inside enclosure is larger than the pressure outside) inside the surgical enclosure such as to ensure that air flows primarily from the inside of the enclosure to the outside environment and that the surgical enclosure is properly inflated. In another embodiment, the control-system is configured such as to maintain a specified material tension into the wall of the surgical enclosure, as measured by a separate sensor disposed into the wall or inferred through pressure readings. In another embodiment, the pressure sensor may be a differential pressure sensor and the environment inside the enclosure maintained at a set pressure irrespective of the outside environment pressure, within pre-set minimum and maximum pressure readings (which may be due to, but not necessarily limited to: sensor specifications; classification of the outside environment as extreme such as a high altitude low temperature environment; or other indications).

The air-tube 72 may be made of flexible plastic material layers (e.g., the same material as the enclosure material, polyethylene, PVC, etc.) including walls which are flexible and collapsible. The walls of the air-tube may act as a tubular two-way valve. For example, when the pressure inside the air-tube is larger than outside the tube the air-tube is expanded in an open state allowing air to flow through the tube. Conversely, when the pressure inside the air-tube is smaller than outside the tube the walls of the air-tube are collapsed in a closed state preventing and/or minimizing air flow through the tube.

### The LED Strip Lights and the Camera

The portable surgical system may include a plurality of LED lights disposed such as to illuminate the surgical site and the inside of the surgical enclosure. In an exemplary embodiment the LED lights may be LED strip lights. The LED strip lights may be disposed on the top of the surgical enclosure such as to illuminate the inside of the enclosure and the surgical site. The LED lights may be powered by the battery of the control device 70.

The portable surgical system may further include one or more cameras configured to receive images (e.g., video or stand still) and monitor the surgical-site. The cameras may be connected with a computer thereby enabling the operators to view the images taken by the camera. The cameras may be disposed either inside the enclosure or outside. The cameras and LED lights may be disposed on a frame-attachment-segment configured to be attached to the frame.

### Methods for Setting up the Surgical System

The surgical system disclosed in this application is configured and may be used by operators to perform surgical procedures on the torso, on the arms/hands, and on the legs/feet of a patient. An exemplary embodiment of the present invention also discloses a method for setting up and using the surgical system. The method may include the steps described hereinafter. The operators identify the surgical site to be operated on and disinfect the patient skin over the surgical site. The flexible enclosure is unfolded and disposed over the patient or adjacent to the patient. If the surgical system employs a rigid frame (such as shown in FIGS. 1-16), then the frame is assembled by connecting the modular segments and the flexible enclosure is attached to the frame via the attachment-means. If the surgical system employs an inflatable-structure (such as shown in FIGS. 17-20), then the inflatable-structure may be inflated via the pressurized gas cartridge thereby bringing the enclosure into desired shape. The surgical enclosure is disposed over the patient so that the incise drape is disposed over the surgical site and the enclosure is attached to the patient via adhesive surrounding the drapes. The environmental control system is assembled, attached to the enclosure, and engaged so as to control air pressure and environment inside the enclosure. The enclosure and the frame may be further secured/affixed over the patient body (and/or to the ground) via affixing means such as straps, tapes, hooks, etc. Materials, devices and instruments may be introduced into the enclosure via the material ports or the arm ports. Tubes and lines of medical instruments may be inserted into the enclosure via the line ports. The environment inside the surgical enclosure attains the required pressure and inflation. At this point, operators insert arms through the sleeves inside the enclosure, may apply gloves, may remove the drapes off the surgical site, may make incisions through the surgical drapes and may perform the surgical procedures.

The methods described herein are not limited to the specific steps and sequence of steps described above. The skilled artisan would recognize that the procedures/steps described herein can be performed in different sequences without departing from the spirit of the invention. The skilled artisan would recognize that many variations can be made to the steps and procedures described herein without departing from the spirit of the invention.

### Portable, Packed, Folded System Suitable for use in Many Environments

The portable surgical systems disclosed herein address both challenges of patient and operator intraoperative exposure to infectious risks. The surgical system ensures that the surgical site is kept in a relatively sterile state (e.g., as sterile as feasible under the conditions) by preventing contaminants from the outer environment (i.e. outside of the surgical enclosure) to reach the surgical site. When used for performing surgery on the torso, the surgical system is configured to ensure that contaminants on the patient body are not reaching the surgical site since, except for the surgical-site, all surface areas of patient body are kept outside of the enclosure. The surgical system provides a barrier protecting operators from exposure to contaminants (e.g., blood, pus, etc.) generated during the surgery inside the enclosure.

The surgical system is configured to be used for performing surgery outdoors such as on wounded soldiers in the field, on inhabitants of remote regions, during rescue operations in wilderness, and in environments which lack the sterility of a hospital operating room (e.g., tents, cottages, residential rooms, non-operating rooms in hospitals, etc.). The surgical system includes batteries configured to provide power to the environmental control system and other devices which may be needed during the surgery. Thus, the surgical system does not require access to electrical grid.

Prior to use, the surgical system is configured to be packed into a portable bag (e.g., backpack) so as to be easy to carry in the field. While packed, the surgical enclosure may be folded like a surgical gown while the frame may be disassembled into its modules. The surgical system is configured to be light, ergonomic and easy to install.

The surgical enclosure 1 is configured to be single use (i.e., after use it will be discarded) while the frame 2 and the external-air-supply system may be used multiple times.

Embodiments of the invention are described herein with reference to figures and illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

The aspects of the invention in this application are not limited to the disclosed operations and sequence of operations. For instance, operations may be performed by various elements and components, may be consolidated, may be omitted, and may be altered without departing from the spirit and scope of the present invention.

The portable surgical systems disclosed herein may include alternate or additional sections which could be added based on procedural needs, such as to accommodate additional instrument trays or users. The above embodiments presented in this disclosure merely serve as exemplary embodiments and it will be apparent to those skilled in the art that various modifications and variations can be made in the present invention without departing from the spirit or scope of the invention.

The terminology used herein is for the purpose of describing particular embodiments only and is not intended to be limiting of the present disclosure. The inventions herein may be embodied in many different forms and should not be construed as limited to the exemplary embodiments set forth herein. Rather, these exemplary embodiments are provided so that this disclosure is thorough, and will fully convey the scope of the invention to skilled artisans.

The following references are incorporated hereinafter as if fully set forth herein: PCT international patent application no. PCT/US 2017/04226 titled “Ultraportable system for intraoperative isolative and regulation of surgical site environments”; PCT international patent application no. PCT/US 2019/032148 titled “Sterile sleeves for portable surgical systems”; PCT international patent application no. PCT/US 2020/032280 titled “Systems and methods for intraoperative isolation and control of surgical site environments”; and PCT international patent application no. PCT/US 2019/051502 titled “Data analytics and interface platform for portable surgical enclosure”.

1. A portable surgical system for performing surgery on a patient, the system comprising:
(a). a flexible surgical enclosure configured to be attached to the body of a patient, the enclosure comprising:
an incise-drape configured to be disposed on the torso of the patient so as to cover a torso-surgical-site of the patient if surgery is needed on the torso-surgical-site;
a patient-limb-port configured to enable the patient to insert an arm or a leg into the enclosure so that a limb-surgical-site is disposed inside the enclosure if surgery is needed on patient's arm or leg;
one or more arm-ports enabling an operator to access and to perform surgery on the torso-surgical-site or on the limb-surgical-site disposed inside the enclosure; and
one or more transparent-material-layers enabling the operator to view the torso-surgical-site or the limb-surgical-site during the surgery;
(b). a frame attached to the flexible surgical enclosure configured to provide stability to the flexible surgical enclosure; and
(c). an environmental air control system configured to supply and automatically control air flow and pressure inside the enclosure such as to ensure a sterile environment inside the enclosure and over the surgical sites, wherein the automatic control is based on sensor readings from the enclosure
wherein when attached to the enclosure the frame is configured to provide a tension over an axial length of the enclosure, via one or more tensioned elements of the frame configured to change shape when outside forces are applied and to provide spring-like resistance/forces, and to create inside the enclosure an operating volume enabling operators to perform surgery on the surgical site.

2. The portable surgical system of claim 1, wherein the surgical enclosure comprises an adhesive-surface disposed around the incise-drape and attached to the patient around the surgical-site so as to create a seal; and wherein, upon removal of the incise-drape the surgical-site of the patient becomes included inside the enclosure and becomes accessible by the operator from the inside of the enclosure whereas other surface areas of the patient are disposed outside the enclosure.

3. The portable surgical system of claim 1, wherein the surgical enclosure further comprises a fluids-reservoir configured to collect the unwanted blood and fluids generated in the enclosure during surgery; wherein the fluids-reservoir is disposed on the lower part of the enclosure and is made as a fold of the enclosure material.

4. The portable surgical system of claim 1, wherein the environmental-control-system comprises:
an air-supply-system comprising a fan, an air-filter, and a control-system;
an air-tube disposed at least partially inside the enclosure and configured to receive air from the air-supply-system; and
wherein the air-tube comprises one or more outlets disposed inside the enclosure and configured to generate air-flow over the surgical site.

5. The portable surgical system of claim 4, wherein the air-supply-system further comprises a pressure sensor configured to measure the pressure inside the enclosure; and wherein the control-system is configured to receive a series of pressure readings from the pressure sensor and to control the air-supply-system such as to adjust the air pressure and air-flow in the enclosure to desired values.

6. The portable surgical system of claim 5, wherein the pressure-sensor is connected to the enclosure via a pressure-tube attached on the enclosure so as to connect the enclosure environment with the pressure-sensor.

7. The portable surgical system of claim 4, wherein the air-tube is made from flexible and collapsible material such as to allow the air-tube to function as a tubular one-way valve and the air-tube closes when the air-supply-system is turned off to minimize air loss from the surgical system.

8. The portable surgical system of claim 1, wherein the patient-limb-port is configured to have an adjustable size so as to accommodate arms or legs of different sizes, from small size to large size compared to average adult limb sizes, while ensuring a suitable air seal into the surgical enclosure.

9. The portable surgical system of claim 1 further comprising one or more material ports configured to enable operators to move materials and instruments in and out of the enclosure, wherein at least one of the material ports comprises magnetic strips enabling the closing and opening of the material ports.

10. The portable surgical system of claim 1 wherein the surgical enclosure comprises one or more folds configured to enable operators to adjust the size of the enclosure.

11. A portable surgical system for performing surgery on a patient, the system comprising:
(a). a flexible surgical enclosure configured to be disposed so as to encompass a surgical site of the patient and to enable an operator to perform surgery on the surgical site through the enclosure, the flexible surgical enclosure comprising one or more flexible transparent layers;
(b). a frame attached to the flexible surgical enclosure, and
(c). an environmental control system configured to supply and control air flow inside the enclosure such as to ensure a sterile environment over the surgical site;
wherein when attached to the enclosure the frame is configured to provide a tension over an axial length of the enclosure, via one or more tensioned elements of the frame configured to change shape when outside forces are applied and to provide spring-like resistance/forces, and to create inside the enclosure an operating volume enabling operators to perform surgery on the surgical site.

12. The portable surgical system of claim 11,
wherein the frame has a loop shape comprising two rigid spacer-segments interspaced by two flexible tensioner-segments,
wherein the tensioner-segments are configured to bend so that the frame assumes essentially a saddle shape when forces are applied to the mid-points of the two tensioner-segments;
wherein a mid-point of the first tensioner-segment is attached to a front axial-end of the enclosure and a mid-point of the second tensioner-segment is attached to a back-axial-end of the enclosure;
wherein the tension over the axial length of the enclosure is generated by the tensions in the bent tensioner-segments.

13. The portable surgical system of claim 12 wherein, while deployed for operation, the flexible enclosure attached to the frame acts on the frame so as to generate tension into the frame and to keep the frame into the saddle shape comprising bent tensioner-segments.

14. The portable surgical system of claim 11 wherein the frame is a modular frame comprising a plurality of segments configured to connect with each other such as to form the loop shaped frame.

15. The portable surgical system of claim 11 wherein the enclosure is attached to the frame via a plurality of attachment-means of adjustable length, wherein a width of the enclosure is adjusted by adjusting a length of the attachment-means.

16. The portable surgical system of claim 11 wherein the enclosure is attached to the frame via a plurality of attachment-means of adjustable length, wherein the inner dimensions of the enclosure and the tensions in the enclosure are configured to be adjusted by adjusting a length of the attachment-means.

17. The portable surgical system of claim 14 wherein at least some of the segments of the frame are configured to have adjustable lengths so that an operator can adjust the dimensions of the frame by adjusting the lengths of the segments.

18. The portable surgical system of claim 11 further comprising one or more of the following: one or more lights configured to illuminate the surgical-site and one or more cameras configured to image the surgical-site.

19. The portable surgical system of claim 18 wherein the one or more lights are LED strip lights disposed on the enclosure or incorporated into the enclosure.

20. The portable surgical system of claim 18 further comprising a frame-attachment-segment configured to be attached to the frame, wherein the one or more lights or the one or more cameras are attached to the frame-attachment-segment.

21. A portable surgical system for performing surgery on a patient, the system comprising:
(a). a flexible surgical enclosure configured to be attached to the body of a patient, the enclosure comprising:
an incise-drape configured to be disposed on the torso of the patient so as to cover a torso-surgical-site of the patient if surgery is needed on the torso-surgical-site;
a patient-limb-port configured to enable the patient to insert an arm or a leg into the enclosure so that a limb-surgical-site is disposed inside the enclosure if surgery is needed on patient's arm or leg;
one or more arm-ports enabling an operator to access and to perform surgery on the torso-surgical-site or on the limb-surgical-site disposed inside the enclosure; and
one or more transparent-material-layers enabling the operator to view the torso-surgical-site or the limb-surgical-site during the surgery;
(b). an inflatable-structure attached to the flexible surgical enclosure and configured to be inflated to an inflatable-structure-pressure higher than the pressure inside the surgical enclosure, wherein when in an inflated state the inflatable-structures provides shape, support and stability to the flexible surgical enclosure;
wherein the inflatable-structure has a saddle-shape including two bent sections disposed opposite to each other and two straigh sections disposed on the side of the enclosure and opposite to each other; and
(c). an environmental air control system configured to supply and control air flow and pressure inside the enclosure such as to ensure a sterile environment inside the enclosure and over the surgical sites.

22. The portable surgical system of claim 21 wherein the inflatable-structure is made of flexible material, is incorporated into the surgical enclosure and is foldable together with the surgical enclosure.

23. The portable surgical system of claim 21 further comprising a pressurized gas cartridge connected to the inflatable-structure, wherein the gas cartridge is configured to inflate the inflatable-structure to the desired inflatable-structure-pressure upon the activation of a trigger-device.

24. A portable surgical system for performing surgery on a patient, the system comprising:
(a). a flexible surgical enclosure configured to be attached to the body of a patient, the enclosure comprising:
an incise-drape configured to be disposed on the torso of the patient so as to cover a torso-surgical-site of the patient if surgery is needed on the torso-surgical-site;
a patient-limb-port configured to enable the patient to insert an arm or a leg into the enclosure so that a limb-surgical-site is disposed inside the enclosure if surgery is needed on patient's arm or leg;
one or more arm-ports enabling an operator to access and to perform surgery on the torso-surgical-site or on the limb-surgical-site disposed inside the enclosure; and
one or more transparent-material-layers enabling the operator to view the torso-surgical-site or the limb-surgical-site during the surgery;
(b). an inflatable-structure attached to the flexible surgical enclosure and configured to be inflated to an inflatable-structure-pressure higher than the pressure inside the surgical enclosure, wherein when in an inflated state the inflatable-structures provides shape, support and stability to the flexible surgical enclosure;
(c). an environmental air control system configured to supply and control air flow and pressure inside the enclosure such as to ensure a sterile environment inside the enclosure and over the surgical sites;
wherein the inflatable-structure comprises two or more rib-air-beams and two base-beams.

25. The portable surgical system of claim 7, wherein the air-tube is made from two flaps composed of flexible and collapsible materials, with the top flap made either lighter, or of a different thickness, or of different flexibility than the bottom flap material, such as to allow the air-tube to function as a tubular one-way valve whereas the top flap collapses under a certain pressure differential between the inside of the enclosure and the outside environment such as to minimize air loss.

26. The portable surgical system of claim 3, wherein the fluids-reservoir comprises a ruler or other visual markings to aid the operator in estimating fluid volume in the fluids-reservoir.

27. The portable surgical system of claim 3, wherein the fluids-reservoir comprises a sensor connected to a computer through wireless or wired means to monitor the fluid loss during the procedure.

28. The portable surgical system of claim 27, wherein the system further comprises an alarm to indicate to the operator when certain levels of fluid loss have been reached during the procedure.
